(19) United States
(12) Patent Application Publication
(10) Pub. No.: US 2021/0219852 A1
(43) Pub. Date: Jul. 22, 2021

(54) Self-Calibrating, Cuffless, and Non-Invasive Blood Pressure Monitor

(71) Applicant: The Trustees of Columbia University in the City of New York, New York, NY (US)

(72) Inventors: David COLBURN, New York, NY (US); Samuel K. SIA, New York, NY (US)

(73) Assignee: The Trustees of Columbia University in the City of New York, New York, NY (US)

(21) Appl. No.: 17/204,352

(22) Filed: Mar. 17, 2021

Related U.S. Application Data
(63) Continuation of application No. PCT/US2019/051431, filed on Sep. 17, 2019.
(60) Provisional application No. 62/840,969, filed on Apr. 30, 2019.
(60) Provisional application No. 62/779,690, filed on Dec. 14, 2018.
(60) Provisional application No. 62/734,573, filed on Sep. 21, 2018.

(51) Int. Cl. A61B 5/021; A61B 5/00; A61B 8/08; A61B 5/055; A61B 5/0245; A61B 5/024

(52) U.S. Cl. CPC A61B 5/02125; A61B 5/681; A61B 8/488; A61B 5/055; A61B 5/7264; A61B 2560/0223; A61B 5/02416; A61B 2562/0219; A61B 2560/0261; A61B 2562/0223; A61B 5/0245

(57) ABSTRACT

The disclosed subject matter includes a wearable device for cuffless blood pressure monitoring that does not require external per-person calibration, such as with a cuff-based measurement device. The embodiment employs photoplethysmography sensors to obtain pulse wave velocity and develops compensation for external pressure influences.

### CROSS-REFERENCE TO RELATED APPLICATIONS

This application is a continuation of International Patent Application No. PCT/US2019/051431 filed Sep. 17, 2019, which claims priority to U.S. Provisional Application No. 62/734,573 filed Sep. 21, 2018, U.S. Provisional Application No. 62/779,690 filed Dec. 14, 2018, and U.S. Provisional Application No. 62/840,969 filed Apr. 30, 2019, each of which is hereby incorporated by reference in its entirety.

### STATEMENT REGARDING FEDERALLY SPONSORED RESEARCH OR DEVELOPMENT

This invention was made with government support under 1UL1TR001873-01 awarded by the National Institutes of Health and 1644869 awarded by the National Science Foundation. The government has certain rights in the invention.

### BACKGROUND

Smart and connected health is a potentially transformative method for predicting early onset of disease that can advance healthcare from reactive to proactive and shift the focus from disease to well-being. However, a major roadblock to achieving this vision is the dearth of user-friendly devices that can track meaningful health data that are accurate, minimally invasive, and unobtrusive. Blood pressure (BP) monitoring is known to provide deep insights into a patient's health for a variety of conditions, including infectious and chronic diseases. Cuffless monitoring is a desirable type of BP monitoring.

### SUMMARY

The disclosed subject matter includes a wearable device for cuffless blood pressure monitoring that does not require external per-person calibration, such as with a cuff-based measurement device. Rather, the embodiments can self-calibrate to ensure accurate blood pressure readings.

Embodiments may include five distinct components to enhance the accuracy of cuffless BP monitoring: (1) a pulse wave detection system, (2) an external pressure compensation system, (3) a processing unit and algorithm for blood pressure tracking, (4) a processing unit and algorithm for calibration, and (5) a processing unit and algorithm for detecting periods of stable blood pressure.

Although the components are listed separately, it should be clear they may be embodied in a same processing unit. For example, all three processing units may be embodied in a single processor or computer.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIG. 1 illustrates PWV acquisition and conversion to blood pressure.

FIG. 2 shows a design for a pulse wave detection system that uses photoplethysmography.

FIG. 3 is a photo photoplethysmographic sensor used on the finger.

FIG. 4 shows example data from the device of FIG. 3.

FIG. 5 shows the data of FIG. 4 after being filtered.

FIG. 6 shows how pulse wave velocity is calculated from the data of FIG. 5.

FIG. 7 shows a block diagram of the sensor fusion algorithm used for altitude tracking.

FIG. 8 shows an example implementation of the hydrostatic pressure compensator.

FIG. 9 shows preliminary data demonstrating the effectiveness of the sensor fusion technique.

FIG. 10 shows filtered photoplethysmography signal and parameterized features.

FIG. 11 shows filtered altitude and parameterized features.

FIG. 12 illustrates how relative altitude at any point can be related to path length traveled as shown in Equation 17.

FIG. 13 shows a block diagram demonstrating use of parameterized features in blood pressure estimation.

FIGS. 14-16 show data related showing how external pressure compensation unit tracks hydrostatic effects.

FIGS. 17-19 show how external pressure compensation can improve systolic pressure estimation accuracy.

FIG. 20 shows block diagram of an example computer system.

### DETAILED DESCRIPTION

The disclosed subject matter includes a wearable device for cuffless blood pressure monitoring that does not require external per-person calibration, such as with a cuff-based measurement device. Rather, the embodiments can self-calibrate to ensure accurate blood pressure readings.

Embodiments may include five distinct components:

1. a pulse wave detection system,

2. an external pressure compensation system,

3. a processing unit and algorithm for blood pressure tracking,

4. a processing unit and algorithm for calibration, and

5. a processing unit and algorithm for detecting periods of stable blood pressure.

The processing units and algorithms need not be separate processors but may be handled using a single processor.

The pulse wave detection system described herein comprises two sensors for recording surrogate proximal and distal signals of the pulse wave, primarily for determination of pulse wave velocity. Pulse wave velocity is the velocity of the pulse wave as it travels down the arterial network and is known to be highly predictive of blood pressure, such as by Equation 1:

BP=K₁ ln(PWV)+K₂ (1)

where K₁ and K₂ are user-specific calibration coefficients. In addition to pulse wave velocity, the pulse signal is used to calculate a number of additional features related to blood pressure.

Pulse wave can be measured by various mechanisms, including any of the following or any combination of the following:

1: two plethysmograph sensors that can be used to measure pulse transit time or pulse wave velocity;

2: one plethysmograph sensor+a sensor that detects heartbeat (e.g. ECG) that can be used to estimate pulse transit time or pulse wave velocity;

3: one or more plethysmograph sensors that can be used to estimate pulse transit time or pulse wave velocity algorithmically from the shape of the waveform;

4: one or more plethysmograph sensor that can be used to estimate transmural pressure algorithmically from the shape of the waveform; or

5: Doppler ultrasound sensor that can be used to measure pulse wave velocity.

6: Magnetic resonance imaging can be used to measure pulse wave velocity and transit time.

Further the plethysmographic sensor may include any suitable configuration including on or a combination of: (1) photoplethysmographic sensor, (2) impedance plethysmographic sensor, (3) strain gauge plethysmographic sensor, (4) magnetic plethysmographic sensor (5) air-displacement plethysmographic sensor, (6) water-displacement plethysmographic sensor, (7) ultrasound based plethysmographic sensor, or (8) an alternative sensor that acquires a non-invasive signal of the pulse wave. In other embodiments, instead of using plethysmographic sensors to measure the velocity of the pulse wave, the velocity of the pulse wave can be estimated using Note that in any of embodiments can be modified to form new embodiments by replacing any recited plethysmographic sensor with a device that measures the velocity of a pulse wave by other means such as the identified Doppler ultrasound.

In an alternative implementation, the proximal signal is acquired using electrocardiogram electrodes and associated conditioning circuitry while the distal signal is acquired using a plethysmographic sensor and associated conditioning circuitry.

The pulse wave detection system can alternatively be comprised of one sensor for recording the pulse wave. Pulse wave velocity can be estimated algorithmically using the signal from a single plethysmographic sensor. The shape of the plethysmogram wave form can be used to detect pulse wave velocity. This may be done with an empirical algorithm, for example using regression or machine learning.

In an alternative embodiment, the single plethysmographic sensor has multiple LEDs of different wavelengths. The pulse wave velocity can be estimated empirically based on the signals generated from the photodetector when excited by the different LEDs.

In another implementation, the wave form of the plethysmogram is used to empirically estimate transmural pressure directly, for example using regression or machine learning. The blood pressure can be derived from the transmural pressure with the relative external pressure.

In all implementations, the photoplethysmography sensor(s) can be placed at various locations. For optimal signal quality, the measurement site should be at a location with an artery near the surface. For the primary implementation, it is expected that the measurement site be at the finger or the wrist, but it could potentially be on another appendage.

FIG. 2 shows an embodiment of the pulse wave detection system 200 using photoplethysmography, example data acquired from these sensors, and the application of these signals in pulse wave velocity acquisition. Two photoplethysmography sensors, 210 and 211 are shown. Each photoplethysmography sensor has a light source 205, such as an LED 205, and a photodetector 204. The photodetector may be a photodiode or a photo transistor, for example. Soft tissue 203 is shown under which is an artery 201. Equations S1-S7 below show the derivation for Equation 1. As mentioned above, embodiments can employ only one photoplethysmographic sensor for a distal signal. The proximal signal may be provided using by an electrocardiogram or some other heartbeat detection device such as an ultrasound sensor to detect heart cycles or an accelerometer to detect heart cycles.

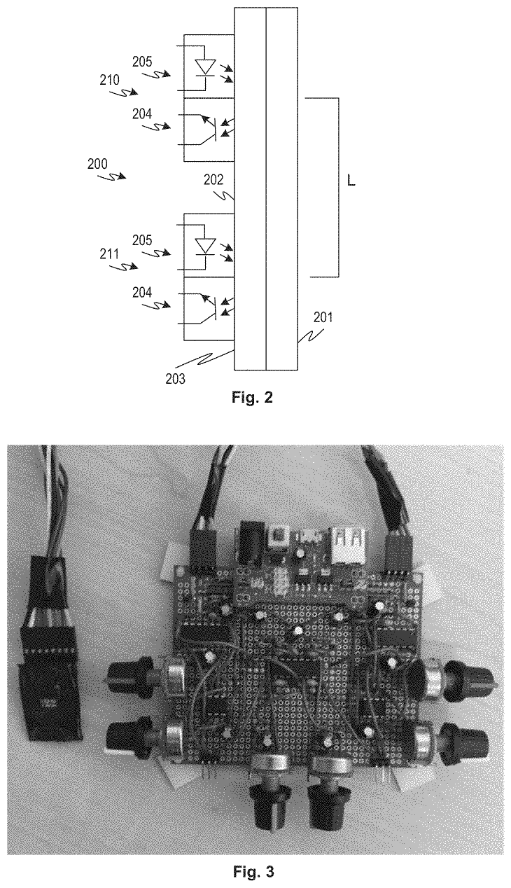

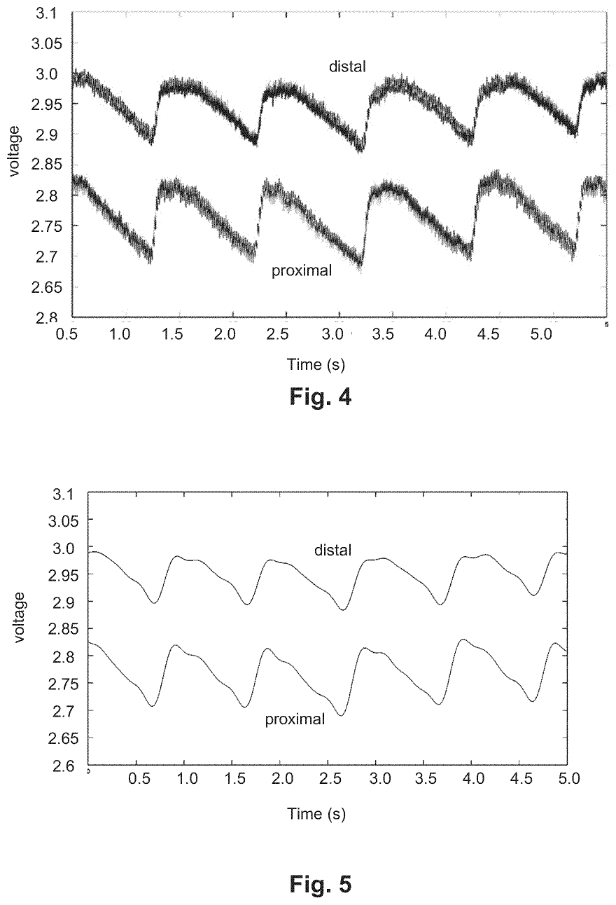

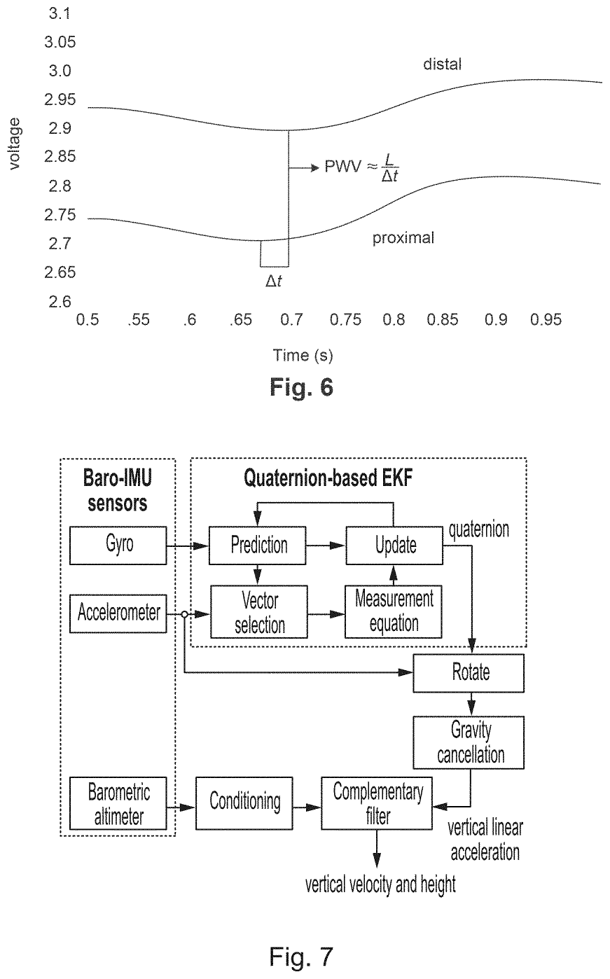

FIG. 3 shows an image of an embodiment of the photoplethysmographic device illustrated in FIG. 2. shows a photo of an embodiment of the device of FIG. 2 applied to the finger. FIG. 4 shows example data acquired from the embodiment of FIGS. 2 and 3. FIG. 5 shows an example of the data of FIG. 4 after low pass filtering. FIG. 6 illustrates how pulse wave velocity is calculated from the data of FIG. 5.

The device may further include an external pressure compensation component. External pressure compensation to account for external pressure applied to the arteries which affects the relationship between pulse wave velocity and blood pressure. To accurately estimate blood pressure based on pulse wave velocity, compensation of the external pressure may be used advantageously.

The external pressure compensation component includes a pressure sensor, for monitoring the contact pressure of the device when applied to the user, and a hydrostatic pressure compensator. The hydrostatic pressure compensator may include an accelerometer, gyroscope, and barometer. The signals from these three sensors are combined using an advanced sensor fusion algorithm that enables tracking altitude changes in real-time with greater accuracy and resolution than possible with the individual sensors alone. The change in altitude relative to the user's heart is used to calculate the hydrostatic pressure contribution by Eqn. 2.

Pₕ=ρgh (2)

Where ρ is the density of the blood, g is the gravitational constant, and h is the altitude relative to the heart. Tracking of hydrostatic pressure is relevant because a difference in elevation of 5 cm between the measurement site and the heart can contribute an error of 3.68 mmHg, more than 50% of the 5 mmHg error allowed by the AAMI measurement standard. Together, the pressure sensor and hydrostatic pressure compensator enable monitoring of the external pressure applied to the arteries and enable more accurate blood pressure measurement.

In an alternative implementation, the external pressure compensation component includes a muscle activation sensor in addition to a contact pressure sensor and hydrostatic pressure compensator. The muscle activation sensor is used to monitor the external pressure applied to the arteries due to muscle contraction.

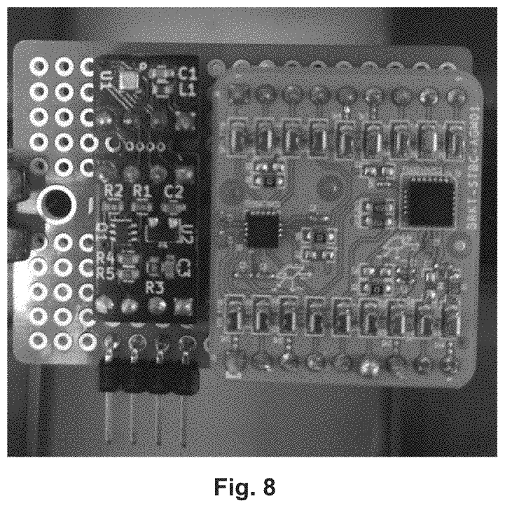

FIG. 7 shows a block diagram of the sensor fusion algorithm used for altitude tracking. FIG. 8 shows an example implementation of the hydrostatic pressure compensator. The example may have a pair of photoplethysmographic sensors, indicates how altitude tracking is performed, and gives preliminary data demonstrating the sensor fusion technique.

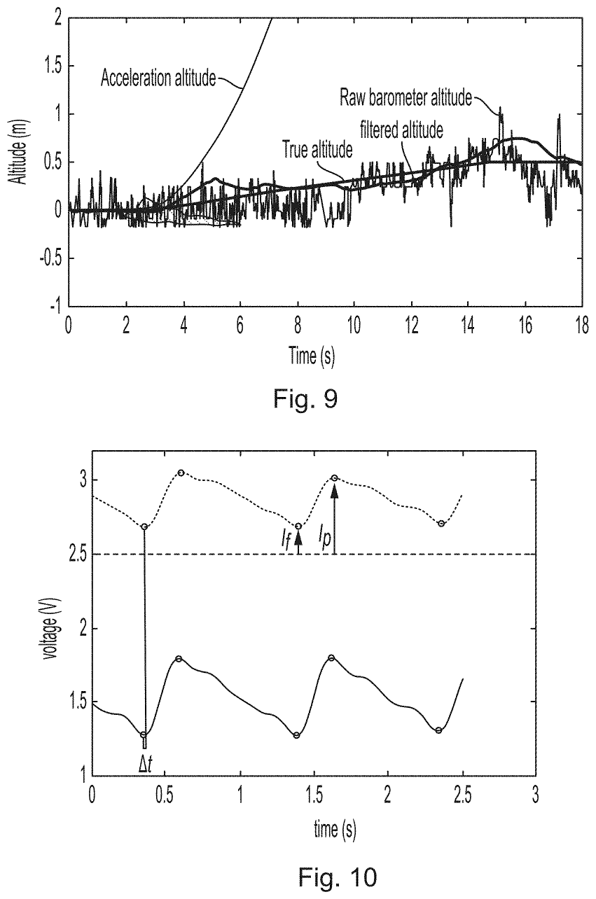

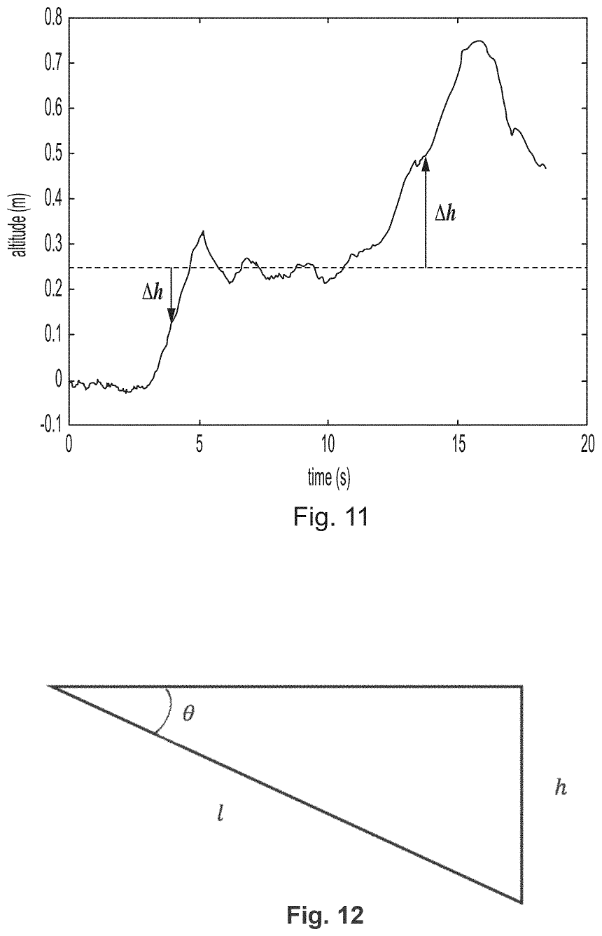

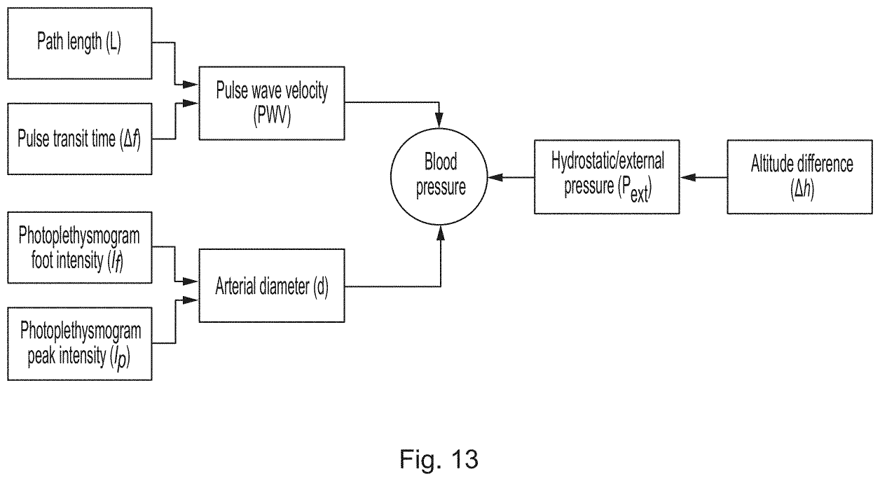

FIG. 9 shows preliminary data demonstrating the effectiveness of the sensor fusion technique. FIG. 10 shows a filtered photoplethysmography signal and parameterized features. FIG. 11 shows filtered altitude and parameterized features. FIG. 12 illustrates how relative altitude at any point can be related to path length traveled as shown in Equation 17. FIG. 13 shows a block diagram demonstrating use of parameterized features in blood pressure estimation.

The embodiment also includes a processing unit that utilizes the data from these sensors to algorithmically track the user's beat-to-beat blood pressure. The sensor data can be related to blood pressure through a number of techniques including, but not limited to, (1) analytical models, (2) linear regression, (3) polynomial regression, (4) machine learning, or (5) a combination of methods.

FIGS. 10, 11, and 13 show examples of the data acquired from the sensors and some of the parameterized features that may be incorporated into the blood pressure tracking algorithm. Derivation S2 (Eqns. S8-S16) is an example of how these features can be utilized to estimate blood pressure. This is not an exhaustive list of features, as some may be found later that prove predictive of blood pressure. Further, Eqn. S16 makes use of only a subset of the identified features. This limitation is because most of the identified features cannot yet be analytically related to blood pressure. As such, this is only a potential method in which these features can be utilized. The optimal algorithm(s) may be optimized for the application.

The embodiment may further include a processing unit that is used for internally calibrating the device to improve the accuracy of the blood pressure estimate. The device is calibrated by monitoring the change in external pressure over a short period of time and the effect on the signals acquired by the different sensors. By assuming blood pressure is constant over that period, the processing unit can calculate the parameters needed to fit or update the algorithm used for beat-to-beat blood pressure tracking.

In one implementation, the amplitude of the plethysmogram waveform is monitored during a period of changing external pressure and is used to calibrate the device. In another implementation, the transit time between different characteristic points in the proximal and distal pulse wave signal is monitored to calibrate the device. In another implementation, a combination of features from the sensors is used to determine the parameters needed to calibrate the device by utilizing a blood pressure tracking algorithm with no bias term.

Derivations S3-S5 demonstrate how each of these implementations may be used to internally-calibrate the device. However, the exact method for internal calibration is dependent on the algorithm that is used for tracking blood pressure. Therefore, this information is offered primarily as a conceptual example.

The calibration procedure may potentially be performed with or without user interaction. In one implementation, the calibration procedure is automatically performed when the device detects a period of changing external pressure and the conditions for assuming constant blood pressure are met. The change in external pressure can be due to changes in contact pressure, hydrostatic pressure, muscle contraction or a combination of these.

In another implementation, the calibration procedure would be performed with user assistance. When the device detects that the conditions for assuming constant blood pressure are met, the user may choose to calibrate the device. The user will then be instructed to perform a series of procedures to perturb the external pressure and thus allow the device to calibrate. For example, if the device is applied to the user's wrist, they may be instructed to slowly raise and lower their arm to alter hydrostatic pressure.

In another implementation, both forms of calibration are used. In this implementation, the automatic method may be the primary means of calibration. However, when a pre-determined period of time has passed since the last user-assisted calibration or a test for calibration quality is not passed, the user may be alerted and instructed to perform a user-assisted calibration procedure.

The embodiments may further include a processing unit that is used to detect periods of constant blood pressure to be used by the internal calibration algorithm. This processing unit monitors the signals from the various sensors and estimates when blood pressure is remaining relatively constant within a pre-determined error bound. To accomplish this, various techniques can be used including, but not limited to, (1) logistic regression classification, (2) support vector machines, (3) neural networks, (4) other machine learning classifiers, or (5) a combination of methods.

The embodiment uses real-time altitude and contact pressure tracking to monitor external pressure and compensate pulse wave velocity-derived blood pressure estimates. The device uses an internal calibration scheme for calibrating a cuffless blood pressure estimation algorithm. Additionally, the device detects periods of stable blood pressure.

The embodiments may be used for ambulatory monitoring of blood pressure for patients at risk for or previously diagnosed with hypertension. The embodiments may be used by patients to monitor their response to antihypertensive drugs. The embodiments could also be used by patients prescribed medication with known blood pressure side-effects to monitor their response. The embodiments could also be used in clinical settings for the continuous, non-invasive monitoring of blood pressure of admitted patients. The embodiments could foreseeably be used as a next-generation fitness tracker that provides continuous blood pressure readings. The embodiments may also be integrated with a larger platform for Smart and Connected Health. This class of devices may be used for improved diagnosis and monitoring of hypertension.

Embodiments may provide user-friendly devices that can track meaningful health data that are accurate, minimally invasive, and unobtrusive. Blood pressure (BP) monitoring provides deep insights into a patient's health for a variety of conditions, including infectious and chronic diseases. Techniques for cuffless BP tracking based on pulse wave velocity (PWV), the velocity of the BP wave, are especially promising. However, current efforts rely on incomplete mathematical models and require repeated per-person calibration, inhibiting adoption. In the present disclosure these models may be updated using an algorithm shown for accurate, calibration-free monitoring of BP using methods that include machine learning. Completing this work may enable a novel class of calibration-free, continuous BP measurements devices and greatly expand the predictive power of smart and connected health.

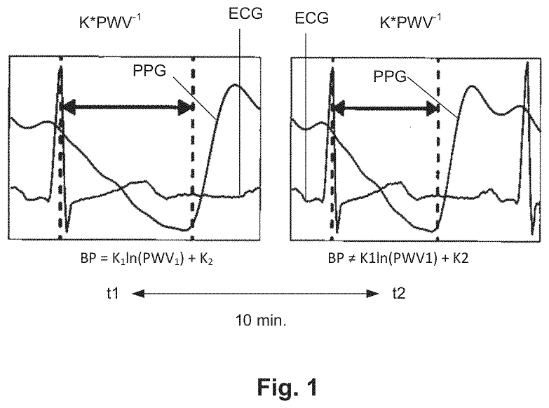

Cuffless BP monitoring from PWV has two fundamental approaches, but they both suffer due to dependence on inadequate models. The first approach estimates BP directly from these simplified models, like the one shown in FIG. 1 where K₁ and K₂ are subject-specific parameters determined through calibration. However, these coefficients are not invariant with time and must be frequently reacquired to maintain tolerable accuracy; thus, devices performing direct estimation using these models fail to accurately track BP as soon as 10 minutes after calibration, as illustrated by FIG. 1. The second approach involves the use of machine learning routines.

Because selection of features optimal for learning is especially challenging, these overly simple models have been used to guide feature selection. Subsequently, the feature vectors have been comprised of characteristics from signals used for PWV acquisition, specifically electrocardiography (ECG) and photoplethysmography (PPG). Despite the use of increasingly sophisticated learning routines, an accurate, calibration-free algorithm has yet to be developed, indicating that these signals are insufficient for accurate BP tracking. Since the development of these simple models, covariates that affect the relationship between BP and PWV, like sensor contact pressure and activity, have been identified and studied. It is believed that accurate and calibration-free BP estimation necessitates tracking PWV and these covariates. Thus, machine learning may be used to develop a calibration-free algorithm for BP monitoring with feature selection guided by an updated model of BP that tracks PWV and key covariates.

We derive an updated model of BP by substituting the latest theoretical and empirical expressions into the equations for conservation of mass and momentum to develop an updated model for BP that is dependent on PWV and relevant covariates. Focus on including covariates that can be tracked using current sensors and have been demonstrated to significantly affect the dependence of BP on PWV. The effects of heartrate, hydrostatic pressure, sensor contact pressure, activity, and ambient temperature may be recorded. This expanded model may be used to inform the selection of features to provide sufficient coverage for accurate estimation of BP. Because the expanded model may account for known confounding variables, it may fit experimental data found in databases like MIMIC II better than the current physical models. However, because parameters like arterial dimensions cannot be easily tracked, application of the model will still depend on patient-specific calibration.

To acquire the data necessary for machine learning, an integrated measurement device uses consumer sensors that can collect signals tracked by the updated physical model. An integrated prototype is preferred because it may ensure the data is consistently acquired and may minimize error attributed to timestamp mismatch. While off-the-shelf components may be used to minimize development difficulties, the device employs a combination of sensors. The device may be comprised of two PPG sensors, for acquisition of PWV in addition to the sensors necessary for covariate tracking, including: a pressure sensor, a 9 degree of freedom sensor, and a temperature sensor. The reference BP may be collected with an FDA approved continuous cuff-based device while the prototype device would concurrently collect the signals of interest. When the updated physical model is applied to this data, it may track BP accurately for a longer period than current equations after initial calibration. Note that instead of two PPG sensors, a proximal signal may be provided from ECG allowing only a single PPG to be used for the distal measurement.

After collecting data from a patient cohort using the device, the identified features may be extracted and divided into learning, validation, and testing sets. Different learning routines may be applied to the training set to generate algorithms for calibration-free estimation of BP. The resulting algorithms will be evaluated using a k-fold Monte Carlo cross-validation scheme to determine which equation performs optimally. Finally, the optimal algorithm to the testing set will be applied. The resulting BP estimates may be statistically compared to the measurements acquired with the commercial monitor to determine the accuracy of the algorithm to unseen data. It is expected that this algorithm, in addition to being more accurate than current equations, eliminates or significantly reduces the need for per-person calibration.

Because it is a complex trait, it may not yet be feasible to track BP completely without calibration. Therefore, if the updated calibration-free equation fails to track BP accurately, the algorithm may be updated to allow for one-time or once-a-day calibration. Alternatively, classification routines may be used to develop a calibration-free algorithm for detecting hypo- or hypertensive events.

While algorithms for tracking BP from PWV have been developed, they fail to account for important covariates, thus limiting their accuracy and necessitating frequent recalibration.

Development and validation of the updated BP estimation algorithm enable cuffless measurement of BP and mark the next step towards the realization of smart and connected health. Further, such an advancement may be used to improve the diagnosis of hypertension and other cardiovascular diseases, diseases which are the leading cause of death in the world and contribute to an economic loss of approximately $250 billion each year in the United States alone. The research may be applied to activities and lesson plans appropriate for various outreach programs, such as Girls' Science Day.

FIG. 2 shows a design for a pulse wave detection system using photoplethysmography sensors FIG. 3 is an image of a sensor implementation with conditioning circuitry FIG. 4 shows example data acquired from this implementation of the device applied to the finger. FIG. 5 shows example of filtered data. FIG. 6 illustrates pulse wave velocity calculation.

FIG. 8 is an image of a hydrostatic pressure compensator implementation. FIG. 7 is a block diagram describing the sensor fusion algorithm used for altitude tracking (Sabatini & Genovese, 2014). FIG. 9 shows preliminary data demonstrating the effectiveness of the sensor fusion technique.

### Derivation S1: Derivation of Common Analytical Blood Pressure Tracking Algorithm

Start with the Moens-Korteweg equation describing pulse wave velocity (PWV) in terms of the elastic modulus of the artery (E), the thickness of the artery (h), blood density (ρ), and the diameter of the artery (d).

$$P W V = \sqrt{\frac{E h}{ρ d}} \qquad ( S1 )$$

Model E as a function of pressure (Pₜᵣₐₙₛ), the elastic modulus at 0 pressure (E₀), and a calibration coefficient (α). Assume that Pₜᵣₐₙₛ is equal to blood pressure (BP)

E=E₀e^{α·P}^{ₜᵣₐₙₛ}=E₀e^{α·BP} (S2)

Substitute equation S2 into equation S1 and rearrange to solve for BP

$$\begin{matrix}P W V = \sqrt{\frac{{E}_{0} h {e}^{α \cdot BP}}{ρ d}} = {e}^{0 .5 α \cdot BP} \times \sqrt{\frac{{E}_{0} h}{ρ d}} & ( S3 ) \\ ln ( PWV ) = ln ( {e}^{0 .5 α \cdot BP} \times \sqrt{\frac{{E}_{0} h}{ρ d}} ) & ( S4 ) \\ ln ( PWV ) = \frac{1}{2} α \cdot BP + ln ( \sqrt{\frac{{E}_{0} h}{ρ d}} ) & ( S5 ) \\ BP = \frac{2}{α} ln ( P W V ) - \frac{2}{α} ln ( \sqrt{\frac{{E}_{0} h}{ρ d}} ) & ( S6 )\end{matrix}$$

Assume that α and the ratio

$$\frac{{E}_{0} h}{ρ d}$$

are constant. Redefine equation S6 in terms of calibration coefficients K₁ and K₂

BP=K₁ ln(PWV)+K₂ (S7)

Derivation S2: Potential Blood Pressure Tracking Algorithm with Additional Features

Start with the Moens-Korteweg equation describing pulse wave velocity (PWV) in terms of the elastic modulus of the artery (E), the thickness of the artery (h), blood density (ρ), and the diameter of the artery (d).

$$P W V = \sqrt{\frac{E h}{ρ d}} \qquad ( S8 )$$

Model E as a function of pressure (Pₜᵣₐₙₛ), the elastic modulus at 0 pressure (E₀), and a calibration coefficient (α).

E=E₀e^{α·P}^{ₜᵣₐₙₛ} (S9)

Define Pₜᵣₐₙₛ as a function of blood pressure (BP) and external pressure (Pₑₓₜ)

Pₜᵣₐₙₛ=BP−Pₑₓₜ (S10)

Substitute equation S10 into equation S9

E=E₀e^{α·BP-α·P}^{ₑₓₜ}=E₀e^{-α·P}^{ₑₓₜ} (S11)

Substitute equation S11 into equation S8. Rearrange to solve for BP

$$\begin{matrix}P W V = \sqrt{\frac{{E}_{0} h {e}^{α \cdot BP} {e}^{- α \cdot {P}_{ext}}}{ρ d}} = \frac{{e}^{0.5 α \cdot BP} {e}^{- 0.5 α \cdot {P}_{e x t}}}{{d}^{0.5}} \times \sqrt{\frac{{E}_{0} h}{ρ}} & ( S12 ) \\ ln ( PWV ) = ln ( \frac{{e}^{0.5 α \cdot BP} {e}^{- 0.5 α \cdot {P}_{e x t}}}{{d}^{0.5}} \times \sqrt{\frac{{E}_{0} h}{ρ}} ) & ( S13 ) \\ ln ( P W V ) = \frac{1}{2} α \cdot BP - \frac{1}{2} α \cdot {P}_{ext} - \frac{1}{2} ln ( d ) + ln ( \sqrt{\frac{{E}_{0} h}{ρ}} ) & ( S14 ) \\ BP = \frac{2}{α} ln ( P W V ) + {P}_{ext} + \frac{d}{α} - \frac{2}{α} ln ( \sqrt{\frac{{E}_{0} h}{ρ}} ) & ( S15 )\end{matrix}$$

Assume that α and the ratio

$$\frac{{E}_{0} h}{ρ}$$

are constant. Redefine equation S6 in terms of calibration coefficients K₁ and K₂

$$B P = {K}_{1} ( ln ( P W V ) + \frac{d}{2} ) + {P}_{ext} + {K}_{2} \qquad ( S16 )$$

### Derivation S3: Calibration by Monitoring Amplitude of Plethysmogram Waveform

Let mean blood pressure (MBP) be equal to the external pressure (Pₑₓₜ) that maximizes the amplitude of the plethysmogram waveform (A(PG))

$$MBP = arg {max}_{{P}_{e x t}} A ( P G ) \qquad ( S17 )$$

Let MBP be a function of pulse wave velocity (PWV), Pₑₓₜ, and calibration coefficients (K₁ and K₂). Solve for specific values of PWV and Pₑₓₜ

MBP=K₁ ln(PWV₁)+K₂+P_{ext,1} (S18)

Solve for K₁

$${K}_{1} = \frac{M B P - {P}_{ext , 1} - {K}_{2}}{ln ( P W {V}_{1} )} \qquad ( S19 )$$

Perturb Pₑₓₜ and measure PWV response. Substitute values into equation S18

MBP=K₁ ln(PWV₂)+K₂+P_{ext,2} (S20)

Substitute equation S19 into equation S20

$$M B P = \frac{M B P - {P}_{ext , 1} - {K}_{2}}{ln ( P W {V}_{1} )} ln ( P W {V}_{2} ) + {K}_{2} + {P}_{ext , 2} \qquad ( S21 )$$

Solve for K₂

$${K}_{2} = \frac{M B P - {P}_{ext , 2} - \frac{( M B P - {P}_{ext , 1} ) ln ( P W {V}_{2} )}{ln ( P W {V}_{1} )}}{1 - \frac{ln ( P W {V}_{2} )}{ln ( P W {V}_{1} )}} \qquad ( S22 )$$

Substitute equation S22 into equation S19 and solve for K₁

$${K}_{1} = \frac{M B P - {P}_{ext , 1}}{ln ( P W {V}_{1} )} - \frac{\begin{matrix}MBP - {P}_{ext , 2} - \\ \frac{( M B P - {P}_{ext , 1} ) ln ( P W {V}_{2} )}{ln ( P W {V}_{1} )}\end{matrix}}{( 1 - \frac{ln ( P W {V}_{2} )}{ln ( P W {V}_{1} )} ) ln ( P W {V}_{1} )} \qquad ( S23 )$$

### Derivation S4: Calibration by Monitoring Timing of Plethysmogram Waveform

Let diastolic blood pressure (DBP) be equal to the external pressure (Pₑₓₜ) that maximizes the foot-measured pulse transit time (PTT_{ƒ})

$$DBP = arg {max }_{{P}_{e x t}} {PTT}_{f} \qquad ( S24 )$$

Let systolic blood pressure (SBP) be equal to Pₑₓₜ that maximizes the peak-measured pulse transit time (PTTₚ)

$$SBP = arg {max}_{{P}_{e x t}} {PTT}_{p} \qquad ( S25 )$$

Let DBP be a function of pulse wave velocity (PWV), Pₑₓₜ, and calibration coefficients (K₁ and K₂). Solve for specific values of PWV and Pₑₓₜ

DBP=K₁ ln(PWV₁)+K₂+P_{ext,1} (S26)

Solve for K₁

$${K}_{1} = \frac{D B P - {P}_{ext , 1} - {K}_{2}}{ln ( P W {V}_{1} )} \qquad ( S27 )$$

Perturb Pₑₓₜ and measure PWV response. Use values to solve for K₂

$${K}_{2} = \frac{D B P - {P}_{ext , 2} - \frac{( D B P - {P}_{ext , 1} ) ln ( P W {V}_{2} )}{ln ( P W {V}_{1} )}}{1 - \frac{ln ( P W {V}_{2} )}{ln ( P W {V}_{1} )}} \qquad ( S28 )$$

Substitute equation 5 into equation 4 to solve for K₁

$${K}_{1} = \frac{D B P - {P}_{ext , 1}}{ln ( P W {V}_{1} )} - \frac{\begin{matrix}DBP - {P}_{ext , 2} - \\ \frac{( D B P - {P}_{ext , 1} ) ln ( P W {V}_{2} )}{ln ( P W {V}_{1} )}\end{matrix}}{( 1 - \frac{ln ( {PWV}_{2} )}{ln ( {PWV}_{1} )} ) ln ( P W {V}_{1} )} \qquad ( S29 )$$

Repeat the procedure to calibrate for SBP

$$\begin{matrix}S B P = {K}_{3} ln ( P W {V}_{1} ) + {K}_{4} + {P}_{ext , 1} & ( S30 ) \\ {K}_{3} = \frac{S B P - {P}_{ext , 1} - {K}_{4}}{ln ( {PWV}_{1} )} & ( S31 ) \\ {K}_{4} = \frac{S B P - {P}_{e x t , 2} - \frac{( S B P - {P}_{ext , 1} ) ln ( P W {V}_{2} )}{ln ( P W {V}_{1} )}}{1 - \frac{ln ( P W {V}_{2} )}{ln ( P W {V}_{1} )}} & ( S32 ) \\ {K}_{3} = \frac{S B P - {P}_{ext , 1}}{ln ( {PWV}_{1} )} - \frac{\begin{matrix}SBP - {P}_{e x t , 2} - \\ \frac{( S B P - {P}_{ext , 1} ) ln ( P W {V}_{2} )}{ln ( P W {V}_{1} )}\end{matrix}}{( 1 - \frac{ln ( {PWV}_{2} )}{ln ( PW {V}_{1} )} ) ln ( P W {V}_{1} )} & ( S33 )\end{matrix}$$

Derivation S5: Calibration from Unbiased Equation

Let diastolic blood pressure (DBP) be described by a function of pulse wave velocity (PWV), external pressure (Pₑₓₜ), and calibration coefficients (K₁ and K₂) that does not have a bias term.

$$D B P = {K}_{1} {e}^{- \frac{{K}_{2}}{P W {V}_{1}}} + {P}_{ext} \qquad ( S34 )$$

Perturb Pₑₓₜ and measure the effect on PWV. Repeat for three different measurements. Simultaneously solve the three equations to find current DBP and calibration coefficients.

$$\begin{matrix}DBP = {K}_{1} {e}^{- \frac{{K}_{2}}{P W {V}_{1}}} + {P}_{{ext}_{1}} & ( S35 ) \\ DBP = {K}_{1} {e}^{- \frac{{K}_{2}}{P W {V}_{2}}} + {P}_{{ext}_{2}} & ( S36 ) \\ DBP = {K}_{1} {e}^{- \frac{{K}_{2}}{P W {V}_{3}}} + {P}_{{ext}_{3}} & ( S37 )\end{matrix}$$

Repeat analysis with SBP to calibrate algorithm

$$\begin{matrix}S B P = {K}_{3} {e}^{- \frac{{K}_{4}}{P W {V}_{1}}} + {P}_{{ext}_{1}} & ( S38 ) \\ SBP = {K}_{3} {e}^{- \frac{{K}_{4}}{P W {V}_{2}}} + {P}_{{ext}_{2}} & ( S39 ) \\ SBP = {K}_{3} {e}^{- \frac{{K}_{4}}{P W {V}_{3}}} + {P}_{{ext}_{3}} & ( S40 )\end{matrix}$$

The embodiments have the following characteristics:

The embodiments include ones in which an external pressure compensation unit for tracking the effect of contact pressure, hydrostatic pressure, and, potentially, smooth muscle contraction. This accounts for the effects of external pressure which may negatively impact the accuracy of the BP estimate.

The disclosed subject matter includes an internal calibration scheme to update the BP tracking algorithm. This allows the device to be calibrated for improved accuracy for individual users which is distinct from external measurement for a one-point calibration.

The disclosed subject matter includes embodiments which contains a mechanism for detecting when BP is stable to allow for internal calibration. This predicts when BP is stable.

The disclosed subject matter includes embodiments in which two photoplethysmography sensors are used and separated by a known distance to estimate local pulse wave velocity. Other embodiments may use ECG as the proximal signal and photoplethysmography as the distal signal to calculate pulse transit time. In these cases, the path length may be inferred to apply the algorithm.

The disclosed subject matter includes embodiments in which one plethysmography sensor is used to estimate pulse wave velocity, transmural pressure, or blood pressure.

The disclosed subject matter includes an external pressure compensation unit to account for the effects of external pressure.

The disclosed subject matter includes embodiments with an internal calibration scheme.

The disclosed subject matter includes embodiments that detect when BP is stable for internal calibration.

### Methods and Results

The disclosed subject matter provides a process for correcting blood pressure estimates generated from cuffless blood pressure monitors to compensate for error due to the external pressure at the measurement site. The major sources of external pressure considered are hydrostatic pressure, contact pressure, pressure from smooth muscle contraction, and pressure from vasoconstriction. However, this process provides a framework for accounting for any source of external pressure.

Embodiments of the disclosed subject matter include the following 3 elements:

(1) a signal acquisition element,

(2) a signal processing element, and

(3) an external pressure compensation element.
a. The signal acquisition element includes of a collection of sensors used to collect information related to external pressure.

In one implementation, the acquisition system contains an accelerometer, gyroscope, magnetometer, and barometer. The data from these sensors can be used to track the relative altitude of the measurement site compared to the user's heart, thereby enabling hydrostatic pressure compensation.

In a second implementation, the acquisition system contains a force sensitive sensor, such as a force sensitive resistor or a force sensitive capacitor, that can measure the pressure of the device when applied to the user, thus enabling contact pressure compensation.

In a third implementation, the acquisition system contains a muscle activation sensor that is used to monitor external pressure due to muscle contraction.

In a fourth implementation, the acquisition system contains a sensor for monitoring the diameter of the artery at the measurement site to allow for compensation of external pressure due to vasoconstriction.

Another implementation contains multiple sensors to enable tracking a combination of external pressure sources.

(2) The signal processing element is used to process the data from the different sensors such that they can be used to correct blood pressure estimates. For hydrostatic pressure, data from the accelerometer, gyroscope, magnetometer, and barometer are combined using an advanced sensor fusion algorithm that enables tracking altitude changes in real-time with greater accuracy and resolution than possible with the individual sensors alone. The sensor fusion technique is illustrated by Eqn. 1A where relative altitude at the measurement site (h*) is given by a function of readings from the accelerometer ( [symbol] ), gyroscope ( [symbol] ), magnetometer ( [symbol] ), and barometer (p_{baro}). The notation can be simplified by absorbing the signals from these different signals into a single sensor term (sₕ), yielding Eqn. 1B.

h*=ƒ( [symbol] , [symbol] , [symbol] ,p_{baro}) (1A)

h*=ƒ(sₕ) (1B)

This relative altitude can then be used to correct for hydrostatic pressure.

For contact pressure, the signal from the force sensitive sensor is used to calculate the contact force applied to the user. This force is then converted to contact pressure by dividing by the surface area of the force sensitive sensor.

Calculation of contact pressure is illustrated in Eqn. 2A where contact pressure (p_{c}) is given by a function of the signal from the sensor (g(s_{c})) divided by the surface area (A).

$${p}_{c} = \frac{g ( {s}_{c} )}{A} \qquad ( 2 A )$$

Note that A is a constant for a given implementation. To simplify notation, absorb A into the function g to describe p_{c} only in terms the signal from the sensor (s_{c}), yielding Eqn. 3A.

p_{c}=α(s_{c}) (3A)

For muscle contraction and vasoconstriction, signals from the associated sensor are acquired and filtered to remove high frequency noise and baseline drift. As there are currently no analytical models for incorporating data from these sensors, their effect on external pressure and blood pressure error would be corrected using machine learning.

(3) After processing the signals, they can be used to compensate the error due to external pressure. To do so, first let the pressure estimated from the cuffless blood pressure monitor be the transmural pressure (pₜᵣₐₙₛ) (preferably the lone PG), the difference between arterial pressure (pₐ) (arterial pressure is the same as blood pressure) and external pressure (pₑₓₜ). The definition of transmural pressure is illustrated by Eqn. 4A.

pₜᵣₐₙₛ=pₐ−pₑₓₜ (4A)

Next, let the arterial pressure be defined as ‘blood pressure’ (p) and decompose external pressure into hydrostatic pressure, contact pressure, pressure due to muscle contraction, and pressure due to vasoconstriction, given by Eqn. 5A. [we are more accurately stated—measuring the change in Pext because we require a baseline—ideally-call it Pₑₓₜ Signals are acquired by the system (1) and processed in the step 2 device in the system to estimate the change in external pressure. Ptrans using the favorite single plethysmogram; after have that you can get the blood pressure for that cardiac cycle]

pₜᵣₐₙₛ=P−(pₕ+p_{c}+pₘ+pᵥ) (5A)

Rearrange, solving for blood pressure, yielding Eqn. 6A.

p=pₜᵣₐₙₛ+pₕ+p_{c}+pₘ+pᵥ (6A)

The rest of the compensation technique depends on if cuffless monitor utilizes a path-independent or -dependent measure for estimating blood pressure.

In the case of a path-independent variable, first replace pₜᵣₐₙₛ with a function of the path-independent variable (ƒ (θ)), yielding Eqn. 7A.

p=ƒ(ϕ)+pₕ+p_{c}+pₘ+pᵥ (7A)

Next, calculate hydrostatic pressure using Eqn. 8A where h is the relative altitude where θ is measured.

pₕ=ρgh (8A)

Then, substitute Eqn. 3A and Eqn. 8A into Eqn. 7A to yield Eqn. 9A.

p=ƒ(θ)+ρgh+α(s_{c})+pₘ+pᵥ (9A)

Next, let the pressure due to muscle contraction and vasoconstriction be defined by machine learning models with the signal from their associated sensors as the independent variable. Substitute these models into Eqn. 9A to yield Eqn. 10A.

p=ƒ(θ)+ρgh+α(s_{c})+β(sₘ)+γ(sᵥ) (10A)

Finally, note that h is equal to h* if θ and altitude measurement site are the same, yielding Eqn. 11A, an equation for blood pressure with external pressure compensation.

p=ƒ(θ)+ρgh*+α(s_{c})+β(sₘ)+γ(sᵥ) (11A)

An example of a path-independent measure that could be used in this equation is pulse wave velocity (PWV), the velocity of the blood pressure wave as it travel through the arterial network. Substituting θ with PWV yields Eqn. 11B, a method for correcting PWV-derived blood pressure estimates.

p=ƒ(PWV)+ρgh*+α(s_{c})+β(sₘ)+γ(sᵥ) (11B)

In the case of path-dependent measures, additional steps are required. First, define the path-dependent measure (ϕ) as the function of a path-independent measure integrated with respect to path (l), yielding Eqn. 12A.

ϕ=∫₀^{L}g(θ)·dl (12A)

Next, rearrange Eqn. 10A and substitute into Eqn. 12A to yield Eqn. 13A.

ϕ=∫₀^{L}g(ƒ⁻¹(p−(ρgh+α(s_{c})+β(sₘ)+γ(sᵥ))))·dl (13A)

An example of a path-dependent measure that could be used in this equation is pulse arrival time (PAT), the time it takes for the blood pressure wave to travel from the heart to some distal site, commonly the finger tip. Using PAT as the path-dependent measured, Eqn. 13A can be rewritten as Eqn. 13B.

$$P A T = {\int }_{0}^{L} \frac{1}{{f}^{- 1} ( p - ( ρ g h + α ( {s}_{c} ) + β ( {s}_{m} ) + γ ( {s}_{v} ) ) )} \cdot dl \qquad ( 13 B )$$

Evaluating these integrals yield an equation for blood pressure that has been corrected for the effects of external pressure. However, the exact solution depends on the form of the different functions, thus the solutions may vary for different implementations and may need to be numerically calculated.

To illustrate a concrete example of how this could be accomplished analytically for PAT, assume that ƒ is a linear model parameterized by the constants K₁ and K₂. Thus, Eqn. 13B can be rewritten as Eqn. 14A.

$$P A T = {\int }_{0}^{L} \frac{{K}_{1}}{p - ( ρ g h + α ( {s}_{c} ) + β ( {s}_{m} ) + γ ( {s}_{v} ) ) - {K}_{2}} \qquad ( 14 A )$$

Further, assume that the effects due to contact pressure, muscle contraction, and vasoconstriction are negligible. Thus, Eqn. 14A reduces to Eqn. 15A.

$$P A T = {\int }_{0}^{L} \frac{{K}_{1}}{p - ρ g h - {K}_{2}} \qquad ( 15 A )$$

Simplify be defining new constants K₃, K₄, and K₅ to yield Eqn. 16A.

$$P A T = {\int }_{0}^{L} \frac{1}{{K}_{3} \cdot p + {K}_{4} h + {K}_{5}} \cdot dl \qquad ( 16 A )$$

Next note that relative altitude, h, is a function of the distance the wave has traveled (1). Thus, h must be redefined in terms of l. For illustrative purposes, assume that the signal is being measured at the finger such that the wave path is down the arm. Next assume that the path of wave travel is straight (e.g. the arm is fully extended) such that relative altitude at any point can be related to path length traveled using Eqn. 17A.

h=l·sin(θ) (17A)

This relationship is further demonstrated by FIG. 12.

If it is assumed that the angle (θ) of the path does not change during a cardiac cycle, then it can be found by substituting the length of the arm (L), and altitude at the finger (h*), yielding Eqn. 18A.

$$θ = arcsin ( \frac{{h}^{*}}{L} ) \qquad ( 18 A )$$

Now, combine Eqn. 18A and Eqn. 17A and substitute into Eqn. 16A to yield Eqn. 19A.

$$P A T = {\int }_{0}^{L} \frac{1}{{K}_{3} \cdot p + {K}_{4} \cdot \frac{{h}^{*}}{L} \cdot l + {K}_{5}} \cdot dl \qquad ( 19 A )$$

Integrate, define new constants, and rearrange to solve for p, yielding Eqn. 20A.

p=A·h*[exp(B·h*·PAT)−1]⁻¹+C (20A)

This equation can be used to calculate blood pressure using PAT while compensating for the effects of hydrostatic pressure under the given assumptions.

In an alternative implementation, Eqn. 13B can be recasted as a machine learning problem. With machine learning, external pressure compensated blood pressure can be found through Eqn. 21A where the function ƒ is approximated using machine learning techniques and is a function of the signals from the various sensors and PAT.

p=ƒ(sₕ,s_{c},sₘ,sᵥ,PAT) (21A)

Disclosed is a general process for correcting cuffless blood pressure estimates in real time by compensating for external pressure. We have conducted a small pilot study that demonstrates that this technique can significantly improve the accuracy of PAT-derived blood pressure estimates. This claim is supported by 14 through 19. As accuracy is the main roadblock to cuffless blood pressure monitors, this the disclosed process may improve the utility of embodiments of the disclosed subject matter.

This disclosed subject matter may forseeably be used as part of a cuffless blood pressure device to improve its accuracy. Further, it could be used as part of an internal calibration system for cuffless blood pressure monitors.

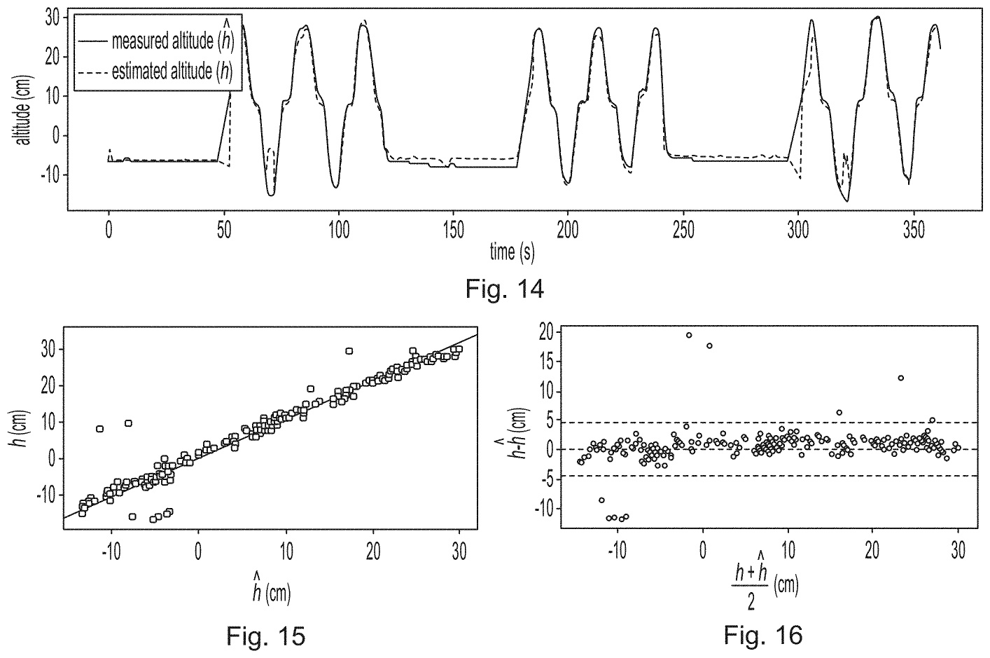

FIGS. 14-16 shows how the external pressure compensation unit tracks hydrostatic effects. A random forest regression model was used to track relative altitude changes using the 10-degree-of-freedom sensor. The time series plot in FIG. 14 shows that these predictions closely follow the reference from the Nexfin. The correlation plot of FIG. 15 shows that the predicted and measured values have a strong correlation (R2=0.97), and the Bland-Altman plot of FIG. 16 shows that there is good agreement between these measures (MAE=1.44±1.51 cm) where the dotted line indicates 95% limits of agreement for the mean difference (grey dotted line).

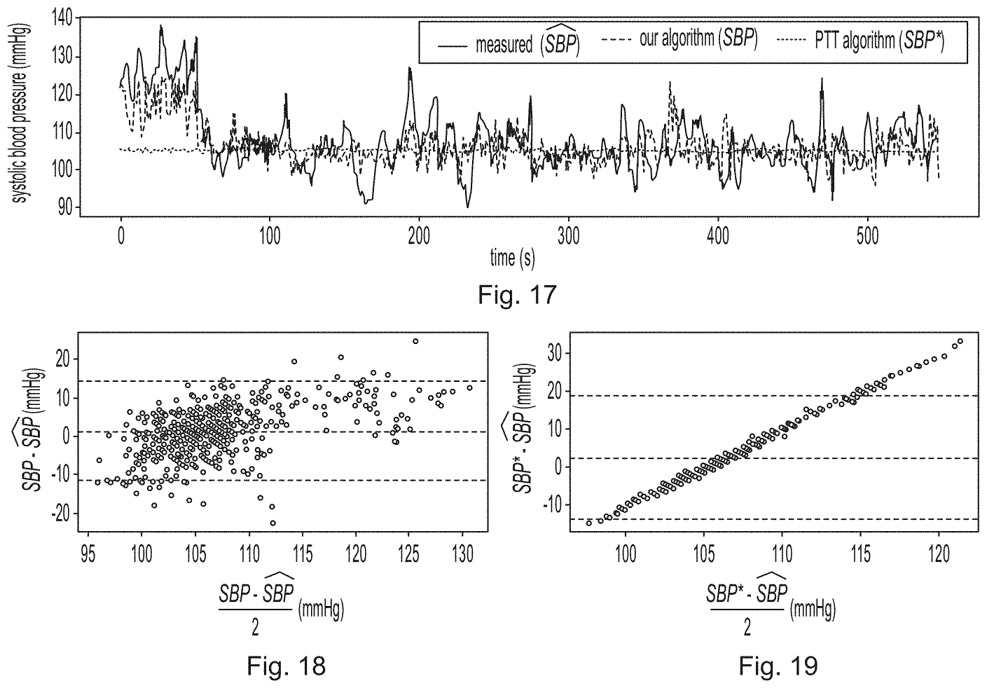

FIGS. 17-19 show how external pressure compensation improves systolic pressure estimation accuracy. A random forest regression model was used to track systolic blood pressure. The time series plot of FIG. 17 shows that the predictions using our technology tracks the reference from the Nexfin better than a competing PTT-based algorithm. The Bland-Altman plot for our algorithm (6.43±5.09 mmHg) (FIG. 18) and the PTT-based algorithm (8.95±8.69 mmHg) (FIG. 19) shows that our estimates have improved accuracy and agreement with the reference (p<0.0001).

It will be appreciated that the modules, processes, systems, and sections described above can be implemented in hardware, hardware programmed by software, software instruction stored on a non-transitory computer readable medium or a combination of the above. For example, a method for measuring blood pressure can be implemented, for example, using a processor configured to execute a sequence of programmed instructions stored on a non-transitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C++, C#.net or the like. The instructions can also comprise code and data objects provided in accordance with, for example, the Visual Basic™ language, LabVIEW, or another structured or object-oriented programming language. The sequence of programmed instructions and data associated therewith can be stored in a non-transitory computer-readable medium such as a computer memory or storage device which may be any suitable memory apparatus, such as, but not limited to read-only memory (ROM), programmable read-only memory (PROM), electrically erasable programmable read-only memory (EEPROM), random-access memory (RAM), flash memory, disk drive and the like.

Furthermore, the modules, processes, systems, and sections can be implemented as a single processor or as a distributed processor. Further, it should be appreciated that the steps mentioned above may be performed on a single or distributed processor (single and/or multi-core). Also, the processes, modules, and sub-modules described in the various figures of and for embodiments above may be distributed across multiple computers or systems or may be co-located in a single processor or system. Exemplary structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a programmable logic device (PLD), programmable logic array (PLA), field-programmable gate array (FPGA), programmable array logic (PAL) device, or the like. In general, any process capable of implementing the functions or steps described herein can be used to implement embodiments of the method, system, or a computer program product (software program stored on a non-transitory computer readable medium).

Furthermore, embodiments of the disclosed method, system, and computer program product may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a very-large-scale integration (VLSI) design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of blood pressure measurement and/or computer programming arts.

Moreover, embodiments of the disclosed method, system, and computer program product can be implemented in software executed on a programmed general purpose computer, a special purpose computer, a microprocessor, or the like.

It is, thus, apparent that there is provided, in accordance with the present disclosure, blood pressure measurement deviced, methods, and systems. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

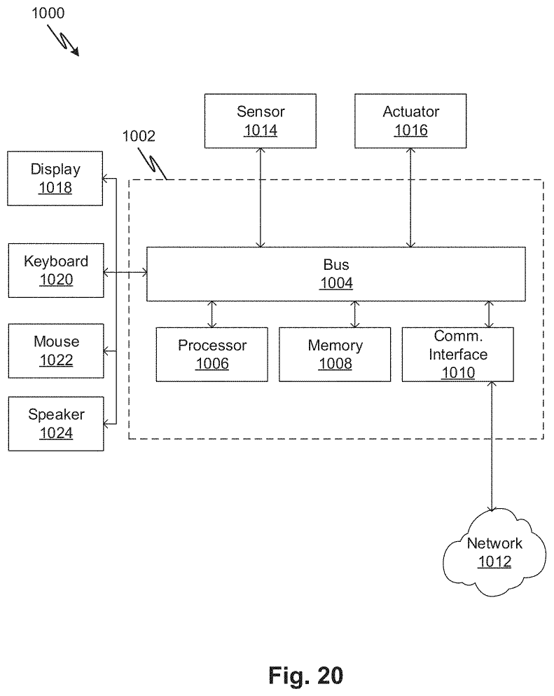

FIG. 20 shows a block diagram of an example computer system according to embodiments of the disclosed subject matter. In various embodiments, all or parts of system 1000 may be embedded in a system such as a diagnostic device. In these embodiments, all or parts of system 1000 may provide the functionality of a controller of the medical treatment device/systems. In some embodiments, all or parts of system 1000 may be implemented as a distributed system, for example, as a cloud-based system.

System 1000 includes a computer 1002 such as a personal computer or workstation or other such computing system that includes a processor 1006. However, alternative embodiments may implement more than one processor and/or one or more microprocessors, microcontroller devices, or control logic including integrated circuits such as ASIC.

Computer 1002 further includes a bus 1004 that provides communication functionality among various modules of computer 1002. For example, bus 1004 may allow for communicating information/data between processor 1006 and a memory 1008 of computer 1002 so that processor 1006 may retrieve stored data from memory 1008 and/or execute instructions stored on memory 1008. In one embodiment, such instructions may be compiled from source code/objects provided in accordance with a programming language such as Java, C++, C#, .net, Visual Basic™ language, LabVIEW, or another structured or object-oriented programming language. In one embodiment, the instructions include software modules that, when executed by processor 1006, provide renal replacement therapy functionality according to any of the embodiments disclosed herein.

Memory 1008 may include any volatile or non-volatile computer-readable memory that can be read by computer 1002. For example, memory 1008 may include a non-transitory computer-readable medium such as ROM, PROM, EEPROM, RAM, flash memory, disk drive, etc. Memory 1008 may be a removable or non-removable medium.

Bus 1004 may further allow for communication between computer 1002 and a display 1018, a keyboard 1020, a mouse 1022, and a speaker 1024, each providing respective functionality in accordance with various embodiments disclosed herein, for example, for configuring a treatment for a patient and monitoring a patient during a treatment.

Computer 1002 may also implement a communication interface 1010 to communicate with a network 1012 to provide any functionality disclosed herein, for example, for alerting a healthcare professional and/or receiving instructions from a healthcare professional, reporting patient/device conditions in a distributed system for training a machine learning algorithm, logging data to a remote repository, etc. Communication interface 1010 may be any such interface known in the art to provide wireless and/or wired communication, such as a network card or a modem.

Bus 1004 may further allow for communication with a sensor 1014 and/or an actuator 1016, each providing respective functionality in accordance with various embodiments disclosed herein, for example, for measuring signals indicative of a patient/device condition and for controlling the operation of the device accordingly. For example, sensor 1014 may provide a signal indicative of a viscosity of a fluid in a fluid circuit in a renal replacement therapy device, and actuator 1016 may operate a pump that controls the flow of the fluid responsively to the signals of sensor 1014.

What is claimed is:

1. A cuffless blood pressure monitor, comprising:
a signal acquisition element including a set of sensors that generate data responsive to transmural and relative external pressure;
the sensors including at least two of a barometer, gyroscope, and an accelerometer;
a processor configured to track altitude and calculate relative external pressure responsively to signals from two or more of said barometer, said gyroscope, and said accelerometer, and output said relative external pressure; and
said processor configured to calculate a transmural pressure responsively to a signal from at least one pulse wave sensor based on the relative external pressure.

2. The monitor of claim 1, wherein the pulse wave sensor includes two plethysmograph sensors that can be used to measure pulse transit time or pulse wave velocity.

3. The monitor of claim 1, wherein the pulse wave sensor includes one plethysmograph sensor and a sensor that detects heartbeat (e.g., ECG) that can be used to estimate pulse transit time or pulse wave velocity.

4. The monitor of claim 1, wherein the pulse wave sensor includes one or more of:
a: one or more plethysmograph sensors that can be used to estimate pulse transit time or pulse wave velocity algorithmically from the shape of a waveform of the pulse wave;
b: one or more plethysmograph sensor that can be used to estimate transmural pressure algorithmically from the shape of the waveform;
c: Doppler ultrasound sensor that can be used to measure pulse wave velocity; or
d: Magnetic resonance imaging can be used to measure pulse wave velocity and transit time.

5. The monitor of claim 1, further comprising a magnetometer wherein said processor is configured to track altitude and calculate relative external pressure responsively to signals from said magnetometer as well as said barometer, gyroscope, and accelerometer.

6. A cuffless blood pressure monitor, comprising:
a device support that can be worn over an artery;
the device support having a pulse wave detection element, an external-pressure processing element, a blood pressure tracking processing element, a calibration processing element, and a stability processing element, wherein said stability processing element is configured to detect periods of stable blood pressure;
the pulse wave detection element including at least one plethysmographic sensor which outputs a wave form;
the external-pressure processing element including a processor to estimate external pressure from both of a contact pressure sensor for measuring contact pressure when applied to a user and a hydrostatic pressure sensor that includes two or more of an accelerometer, a gyroscope, and a barometer, wherein the external-pressure processing element is configured to combine signals from the two or more of an accelerometer, a gyroscope, a barometer to track altitude changes in real-time.

7. The monitor of claim 6, wherein the external-pressure processing element includes the hydrostatic pressure sensor and is configured to combine signals from the two or more of an accelerometer, a gyroscope, a barometer, with signals from a magnetometer to track the altitude changes in real-time.

8. The monitor of claim 7, wherein the hydrostatic pressure sensor includes all three of an accelerometer, a gyroscope, and a barometer.

9. The monitor of claim 6, wherein the at least one plethysmographic sensor is one plethysmographic sensor and wherein the pulse wave detection element also includes a sensor that detects a subject's heartbeat.

10. The monitor of claim 9, wherein the plethysmographic sensor is configured to attach to a subject's wrist or finger and overly an artery and the plethysmographic sensor is located in a single physical element that also contains a force transducer to detect contact pressure.

11. The monitor of claim 6, wherein a relationship between blood pressure and the signals from the sensors is obtained by an analytical algorithm, a linear regression, a polynomial regression, machine learning, or a combination thereof.

12. The monitor of claim 11, wherein the blood pressure and said sensors are related by monitoring the change in external pressure over a predefined period of time and the effect on the signals acquired by the sensors such that blood pressure is constant over the predefined period of time so that the calibration processing element can calculate parameters needed to fit or update the algorithm used for blood pressure tracking.

13. The monitor of claim 12, wherein said relationship between blood pressure and said sensors is obtained when the stability processing element indicates blood pressure is constant over said predefined period of time.

14. The monitor of claim 12, wherein a calibration is automatically begun in response to a change in external pressure.

15. The monitor of claim 13, wherein the calibration processing element outputs instructions on a display indicating steps a user should do to perform a user-assisted calibration.

16. A cuffless blood pressure monitor, comprising:
a device support that can be placed or worn over an artery;
the device support having a pulse wave detection element, an external-pressure processing element, a blood pressure tracking processing element, a calibration processing element, and a stability processing element configured to detect periods of stable blood pressure;
the pulse wave detection element including a single plethysmographic sensor whose output signal is characterized by a wave form, wherein the shape of the wave form is used to obtain pulse wave velocity, transmural pressure, or blood pressure using an empirical algorithm such as is obtained using empirical data which is processed using regression or machine learning;
the external-pressure processing element including a processor to estimate external pressure from both of a contact pressure sensor for measuring contact pressure when applied to a user and a hydrostatic pressure sensor that includes two or more of an accelerometer, a gyroscope, and a barometer, wherein the external-pressure processing element is configured to combine signals from the two or more of the accelerometer, a gyroscope, a barometer to track altitude changes in real-time;
the controller being configured to output a signal indicating an estimate of blood pressure.

17. The monitor of claim 16, wherein the hydrostatic pressure sensor includes all three of an accelerometer, a gyroscope, and a barometer.

18. The monitor of claim 16, wherein a relationship between blood pressure and the signals from the sensors is obtained by an analytical algorithm, a linear regression, a polynomial regression, machine learning, or a combination thereof.

19. The monitor of claim 18, wherein the external-pressure processing element is calibrated by monitoring the change in external pressure over a predefined period of time and the effect on the signals acquired by the sensors where blood pressure is constant over the predefined period of time such that the processing element for detecting periods of stable blood pressure can calculate parameters needed to fit or update the algorithm used for blood pressure tracking.

20. The monitor of claim 19, wherein a calibration is automatically begun in response to a change in external pressure.
